(19) United States
(12) Patent Application Publication
(10) Pub. No.: US 2007/0015148 A1
(43) Pub. Date: Jan. 18, 2007

(54) Gene expression profiles in breast tissue

(76) Inventors: Michael S Orr, Gaithersburg, MD (US); MIchelle Nation, Gaithersburg, MD (US); James C Diggans, Gaithersburg, MD (US); Wen Zeng, Gaithersburg, MD (US)

Correspondence Address: MORGAN LEWIS & BOCKIUS LLP, 1111 PENNSYLVANIA AVENUE NW, WASHINGTON, DC, 20004, US

(21) Appl. No.: 10/470,050

(22) PCT Filed: Jan. 25, 2002
(86) PCT No.: PCT/US02/02176

§ 371 Date: Jul. 25, 2003

Related U.S. Application Data
(60) Provisional application No. 60/263,757, filed on Jan. 25, 2001.
(60) Provisional application No. 60/286,090, filed on Apr. 25, 2001.
(60) Provisional application No. 60/292,517, filed on May 23, 2001.

(51) Int. Cl. C12Q 1/68; A61K 48/00

(52) U.S. Cl. 435/6; 514/44

(57) ABSTRACT

The present invention results from the examination of tissue from breast carcinomas to identify genes differentially expressed between tumor biopsies and normal tissue. The invention includes diagnostic and screening methods using these genes as well as solid supports comprising oligonucleotide arrays that are complementary to or hybridize to the differentially expressed genes.

### RELATED APPLICATIONS

This application claims the priority of U.S. Provisional Application No. 60/263,757, filed Jan. 25, 2001, 60/286,090, filed Apr. 25, 2001, and 60/292,517, filed May 23, 2001, all of which are herein incorporated by reference in their entirety.

### BACKGROUND OF THE INVENTION

One of the most pressing health issues today is breast cancer. In the industrial world, about one woman in every nine can expect to develop breast cancer in her lifetime. In the United States, it is the most common cancer amongst women, with an annual incidence of about 175,000 new cases and nearly 50,000 deaths. Despite an ongoing improvement in our understanding of the disease, breast cancer has remained resistant to medical intervention. Most clinical initiatives are focused on early diagnosis, followed by conventional forms of intervention, particularly surgery and chemotherapy. Such interventions are of limited success, particularly in patients where the tumor has undergone metastasis. There is a pressing need to improve the arsenal of therapies available to provide more precise and more effective treatment in a less invasive way. A promising area for the development of new modalities has emerged from recent understanding of the genetics of cancer.

One model used to characterize breast carcinogenesis asserts that normal cells undergo a multi-step process that broadly includes the steps of hyperplasia, pre-malignant change and in situ carcinoma. Multiple factors lead to a typical cell proliferation followed by carcinoma in situ. Carcinoma in situ is characterized as either ductal or lobular in form with the majority of invasive carcinomas being classified as ductal (85-95%). Among the ductal carcinomas, 15-20% encompass tubular, medullary, mucinous, papillary, adenoid, cystic, metaplastic, apocrine, squamous, secretory, lipid-rich, and cystic hypersecretory while the remaining ductal carcinomas are not specified.

To date, researchers have been able to identify a few genetic alterations believed to underlie tumor development. These genetic alterations include amplification of oncogenes and mutations that result in the loss of tumor suppressor genes. Tumor suppressor genes are genes that, in their wild-type alleles, express proteins that suppress abnormal cellular proliferation. When the gene coding for a tumor suppressor protein is mutated or deleted, the resulting mutant protein or the complete lack of tumor suppressor protein expression may fail to correctly regulate cellular proliferation, and abnormal proliferation may take place, particularly if there is already existing damage to the cellular regulatory mechanism. A number of well-studied human tumors and tumor cell lines have missing or non-functional tumor suppressor genes. Examples of tumor suppressor genes include, but are not limited to, the retinoblastoma susceptibility gene or RB gene, the p53 gene, the deletion in colon carcinoma (DCC) gene and the neurofibromatosis type 1 (NF-1) tumor suppressor gene (Weinberg, Science 254,1138-1146 (1991)). Loss of function or inactivation of tumor suppressor genes may play a central role in the initiation and/or progression of a significant number of human cancers.

Classification of heterogeneous populations of tumor types is a daunting task; yet, studies utilizing gene expression patterns to identify subtypes of cancer have produced initial results (see Perou, C. M. et al., Proc Natl Acad Sci USA 96, 9212-9217 (1999), Golub, T. R. et al., Science 286, 531-7 (1999), Alizadeh, A. A. et al., Nature 403, 503-11 (2000), Alon, U. et al. Proc Natl Acad Sci USA 96, 6745-50 (1999) and Bittner, M. et al., Nature 406, 536-40 (2000)). For example, molecular classification of B-cell lymphoma by gene expression profiling elucidated clinically distinct diffuse large-B-cell lymphoma subgroups (see Alizadeh supra). Stratification of patients based on their distinctive gene expression profiles may allow researchers to precisely group similar patient populations for evaluating chemotherapeutic agents. The more homogenous population of patients decreases the variability of patient-to-patient responses leading to the development of agents capable of eradicating specific subtypes of cancers previously unknown using standard classification techniques.

A study by Martin et al. (Cancer Res 60, 2232-8 (2000)) used a custom microarray composed of 124 genes discovered by differential display associated with either normal breast epithelial cells or from the MDA-MB-435 malignant breast tumor cell line. Using the custom microarray, researchers examined the relationship between expression patterns discovered by clustering a number of genes with clinical stages of breast cancer, indicating that gene expression patterns were capable of grouping breast tumors into distinct categories (Martin et al., supra).

The utilization of gene expression profiles to classify tumors, to identify drug targets, to identify diagnostic markers and/or to gain further insights into the consequences of chemotherapeutic treatments could facilitate the design of more efficacious patient-specific stratagems for treating a variety of cancers. In breast cancer, studies utilizing limited numbers of genes have classified tumors into subtypes based on gene expression profiles, and this study indicated a diversity of molecular phenotypes associated with breast tumors (Perou, C. M. et al., Nature 406, 747-52 (2000).

Although these studies have demonstrated that expression profiling may be used to produce improvements in diagnosis of breast cancer as well as the development of improved therapeutic strategies, further studies are needed as only a small portion of the genome was studied and analyses containing greater numbers of genes will advance our understanding of breast tumors even further. Accordingly, there remains a need in the art for materials and methods that permit a more accurate diagnosis of breast cancer and, in particular, ductal carcinoma. In addition, there remains a need in the art for methods to treat and methods to identify agents that can effectively treat breast cancer. The present invention meets these and other needs.

### SUMMARY OF THE INVENTION

The present invention is based on the discovery of the genes and their expression profiles associated with various types and stages of breast cancer.

The invention includes methods of diagnosing breast cancer in a patient comprising the step of detecting the level of expression in a tissue sample of two or more genes from Tables 1-5; wherein differential expression of the genes in Tables 1-5 is indicative of breast cancer.

The invention also includes methods of detecting the progression of breast cancer. For instance, methods of the invention include detecting the progression of breast cancer in a patient comprising the step of detecting the level of expression in a tissue sample of two or more genes from Tables 1-5; wherein differential expression of the genes in Tables 1-5 is indicative of breast cancer progression. In some preferred embodiments, PCA (Principal Component Analysis) based on all or a portion of the group of 50 genes identified in Table 1 may be used to differentiate between the different stages of breast cancer such as normal versus DCIS (ductal carcinoma in-situ) or DCIS versus microinvasive tissue samples. In some preferred embodiments, one or more genes may be selected from Tables 1, 3, 4 and/or 5.

In some aspects, the present invention provides a method of monitoring the treatment of a patient with breast cancer, comprising administering a pharmaceutical composition to the patient and preparing a gene expression profile from a cell or tissue sample from the patient and comparing the patient gene expression profile to a gene expression from a cell population comprising normal breast cells or to a gene expression profile from a cell population comprising breast cancer cells or to both. In some preferred embodiments, the gene profile will include the expression level of one or more genes in Tables 1-5.

Another aspect of the present invention includes a method of treating a patient with breast cancer, comprising administering to the patient a pharmaceutical composition, wherein the composition alters the expression of at least one gene in Tables 1-5, preparing a gene expression profile from a cell or tissue sample from the patient comprising tumor cells and comparing the patient expression profile to a gene expression profile from an untreated cell population comprising breast cancer cells.

In another aspect, the present invention provides a method of identifying ductal carcinoma in a patient, comprising detecting the level of expression in a tissue sample of two or more genes from Tables 1-5, wherein differential expression of the genes in Tables 1-5 is indicative of ductal carcinoma. In addition, by determining the expression level of two or more genes in the group of genes listed in Tables 1-5, one skilled in the art can differentiate between DCIS and a cribiform type of DCIS that is more prone to microinvasion.

In another aspect, the present invention provides a method of detecting the progression of carcinogenesis in a patient, comprising detecting the level of expression in a tissue sample of two or more genes from Tables 1-5; wherein differential expression of the genes in Tables 1-5 is indicative of breast carcinogenesis. FIGS. 6 and 7 are a graphical representation of how the genes listed in Table 5 cluster with disease stages in breast cancer.

The invention further includes methods of screening for an agent capable of modulating the onset or progression of breast cancer, comprising the steps of exposing a cell to the agent; and detecting the expression level of two or more genes from Tables 1-5. In some embodiments, the breast cancer may be a ductal carcinoma. In some preferred embodiments, one or more genes may be selected from a group consisting of those listed in Tables 1, 3, 4 and/or 5. In some preferred methods, it may be desirable to detect all or nearly all of the genes in the tables.

The invention further includes compositions comprising at least two oligonucleotides, wherein each of the oligonucleotides comprises a sequence that specifically hybridizes to a gene in Tables 1-5 as well as solid supports comprising at least two probes, wherein each of the probes comprises a sequence that specifically hybridizes to a gene in Tables 1-5. In some preferred embodiments, one or more genes may be selected from a group consisting of those listed in Tables 1, 3, 4 and/or 5.

The invention further includes computer systems comprising a database containing information identifying the expression level in breast tissue of a set of genes comprising at least two genes in Tables 1-5 and a user interface to view the information. In some preferred embodiments, one or more genes may be selected from a group consisting of those listed in Tables 1, 3, 4 and/or 5. The database may further include sequence information for the genes, information identifying the expression level for the set of genes in normal breast tissue and cancerous tissue and may contain links to external databases such as GenBank.

Lastly, the invention includes methods of using the databases, such as methods of using the disclosed computer systems to present information identifying the expression level in a tissue or cell of at least one gene in Tables 1-5, comprising the step of comparing the expression level of at least one gene in Tables 1-5 in the tissue or cell to the level of expression of the gene in the database. In some preferred embodiments, two or more genes may be selected from a group consisting of those listed in Tables 1, 3, 4 and/or 5.

### BRIEF DESCRIPTION OF THE DRAWINGS

FIG. 1 is an E-northern showing the expression of topoisomerase II alpha in various tissue types.

FIG. 2 is an E-northern showing the expression of ICBP90 in various tissue types.

FIG. 3 is an E-northern showing the expression of MCT4 gene.

FIG. 4 is an E-northern showing the expression of the frizzled related protein.

FIG. 5 is an E-northern showing the expression of an EST Affy ID AI668620.

FIG. 6 is a PCA of the set of 28 samples using the top 50 genes identified by p-values.

FIG. 7 is a PCA of the set of 33 samples using the top 50 genes and ESTs identified by p-values.

FIG. 8 is a PCA of the set of 91 samples using the top 31 myo-lamina genes and ESTs.

### DETAILED DESCRIPTION OF THE PREFERRED EMBODIMENTS

Many biological functions are accomplished by altering the expression of various genes through transcriptional (e.g., through control of initiation, provision of RNA precursors, RNA processing, etc.) and/or translational control. For example, fundamental biological processes such as cell cycle, cell differentiation and cell death, are often characterized by the variations in the expression levels of groups of genes.

Changes in gene expression also are associated with pathogenesis. For example, the lack of sufficient expression of functional tumor suppressor genes and/or the over expression of oncogene/protooncogenes could lead to tumorgenesis or hyperplastic growth of cells (Marshall, Cell 64, 313-326 (1991); Weinberg, Science, 254, 1138-1146 (1991)). Thus, changes in the expression levels of particular genes (e.g., oncogenes or tumor suppressors) serve as signposts for the presence and progression of various diseases.

Monitoring changes in gene expression may also provide certain advantages during drug screening and development. Often drugs are pre-screened for the ability to interact with a major target without regard to other effects the drugs have on cells. Often such other effects cause toxicity in the whole animal, which prevent the development and use of the potential drug.

Applicants have examined samples from normal breast tissue and from cancerous breast tissue to identify global changes in gene expression between tumor biopsies and normal tissue. These global changes in gene expression, also referred to as expression profiles, provide useful markers for diagnostic uses as well as markers that can be used to monitor disease states, disease progression, drug toxicity, drug efficacy and drug metabolism.

The gene expression profiles described herein were derived from normal and tumor samples from female patients between the ages of 39 and 52 years old, and were from three different ethnic origins (Caucasian, African-American and Asian). Infiltrating Ductal Carcinoma (IDC) patient samples were studied for cancer-related expression, as 85% of the breast cancer patients were afflicted with this form of the disease.

Histological analysis of each tissue sample was performed and samples were segregated into either normal or malignant categories. The normal tissue samples were acquired from neighboring tissue of patients suffering from one of the following disorders: macromastia, mild fibrosis, infiltrating lobular carcinoma, or infiltrating ducal carcinoma, however; each tissue was diagnosed as normal by histological analysis. Samples were also characterized by the type and grade of IDC for each patient sample utilized in the study.

The present invention provides compositions and methods to detect the level of expression of genes that may be differentially expressed dependent upon the state of the cell, i.e., normal versus cancerous. These expression profiles of genes provide molecular tools for evaluating toxicity, drug efficacy, drug metabolism, development, and disease monitoring. Changes in the expression profile from a baseline profile can be used as an indication of such effects. Those skilled in the art can use any of a variety of known techniques to evaluate the expression of one or more of the genes and/or gene fragments identified in the instant application in order to observe changes in the expression profile in a tissue or sample of interest.

Definitions

In the description that follows, numerous terms and phrases known to those skilled in the art are used. In the interest of clarity and consistency of interpretation, the definitions of certain terms and phrases are provided.

As used herein, the phrase “detecting the level of expression” includes methods that quantify expression levels as well as methods that determine whether a gene of interest is expressed at all. Thus, an assay which provides a yes or no result without necessarily providing quantification of an amount of expression is an assay that requires “detecting the level of expression” as that phrase is used herein.

As used herein, oligonucleotide sequences that are complementary to one or more of the genes described herein, refers to oligonucleotides that are capable of hybridizing under stringent conditions to at least part of the nucleotide sequence of said genes. Such hybridizable oligonucleotides will typically exhibit at least about 75% sequence identity at the nucleotide level to said genes, preferably about 80% or 85% sequence identity or more preferably about 90% or 95% or more nucleotide sequence identity to said genes.

“Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The terms “background” or “background signal intensity” refer to hybridization signals resulting from non-specific binding, or other interactions, between the labeled target nucleic acids and components of the oligonucleotide array (e.g., the oligonucleotide probes, control probes, the array substrate, etc.). Background signals may also be produced by intrinsic fluorescence of the array components themselves. A single background signal can be calculated for the entire array, or a different background signal may be calculated for each target nucleic acid. In a preferred embodiment, background is calculated as the average hybridization signal intensity for the lowest 5% to 10% of the probes in the array, or, where a different background signal is calculated for each target gene, for the lowest 5% to 10% of the probes for each gene. Of course, one of skill in the art will appreciate that where the probes to a particular gene hybridize well and thus appear to be specifically binding to a target sequence, they should not be used in a background signal calculation. Alternatively, background may be calculated as the average hybridization signal intensity produced by hybridization to probes that are not complementary to any sequence found in the sample (e.g., probes directed to nucleic acids of the opposite sense or to genes not found in the sample such as bacterial genes where the sample is mammalian nucleic acids). Background can also be calculated as the average signal intensity produced by regions of the array that lack any probes at all.

The phrase “hybridizing specifically to” refers to the binding, duplexing or hybridizing of a molecule substantially to or only to a particular nucleotide sequence or sequences under stringent conditions when that sequence is present in a complex mixture (e.g., total cellular) DNA or RNA.

Assays and methods of the invention may utilize available formats to simultaneously screen at least about 100, preferably about 1000, more preferably about 10,000 and most preferably about 1,000,000 or more different nucleic acid hybridizations.

The terms “mismatch control” or “mismatch probe” refer to a probe whose sequence is deliberately selected not to be perfectly complementary to a particular target sequence. For each mismatch (MM) control in a high-density array there typically exists a corresponding perfect match (PM) probe that is perfectly complementary to the same particular target sequence. The mismatch may comprise one or more bases that are not complementary to the corresponding bases of the target sequence.

While the mismatch(s) may be located anywhere in the mismatch probe, terminal mismatches are less desirable as a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions.

The term “perfect match probe” refers to a probe that has a sequence that is perfectly complementary to a particular target sequence. The test probe is typically perfectly complementary to a portion (subsequence) of the target sequence. The perfect match (PM) probe can be a “test probe”, a “normalization control” probe, an expression level control probe and the like. A perfect match control or perfect match probe is, however, distinguished from a “mismatch control” or “mismatch probe.”

As used herein a “probe” is defined as a nucleic acid, preferably an oligonucleotide, capable of binding to a target nucleic acid of complementary sequence through one or more types of chemical bonds, usually through complementary base pairing, usually through hydrogen bond formation. As used herein, a probe may include natural (i.e., A, G, U, C or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages.

The term “stringent conditions” refers to conditions under which a probe will hybridize to its target subsequence, but with only insubstantial hybridization to other sequences or to other sequences such that the difference may be identified. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH.

Typically, stringent conditions will be those in which the salt concentration is at least about 0.01 to 1.0 M sodium ion concentration (or other salts) at pH 7.0 to 8.3 and the temperature is at least about 30° C. for short probes (e.g., 10 to 50 nucleotide). Stringent conditions may also be achieved with the addition of destabilizing agents such as formamide.

The “percentage of sequence identity” or “sequence identity” is determined by comparing two optimally aligned sequences or subsequences over a comparison window or span, wherein the portion of the polynucleotide sequence in the comparison window may optionally comprise additions or deletions (i.e., gaps) as compared to the reference sequence (which does not comprise additions or deletions) for optimal alignment of the two sequences. The percentage is calculated by determining the number of positions at which the identical subunit (e.g., nucleic acid base or amino acid residue) occurs in both sequences to yield the number of matched positions, dividing the number of matched positions by the total number of positions in the window of comparison and multiplying the result by 100 to yield the percentage of sequence identity. Percentage sequence identity when calculated using the programs GAP or BESTFIT (see below) is calculated using default gap weights.

Homology or identity may be determined by BLAST (Basic Local Alignment Search Tool) analysis using the algorithm employed by the programs blastp, blastn, blastx, tblastn and tblastx (Karlin et al., Proc Natl Acad Sci USA 87, 2264-2268 (1990) and Altschul, J Mol Evol 36, 290-300 (1993), fully incorporated by reference) which are tailored for sequence similarity searching. The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al., (Nature Genet 6, 119-129 (1994)) which is fully incorporated by reference. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., Proc Natl Acad Sci USA 89, 10915-10919, (1992) fully incorporated by reference). Four blastn parameters were adjusted as follows: Q=10 (gap creation penalty); R=10 (gap extension penalty); wink=1 (generates word hits at every wink^{th} position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

Uses of Differentially Expressed Genes

The present invention identifies those genes differentially expressed between normal breast tissue and cancerous breast tissue. One of skill in the art can select one or more of the genes identified as being differentially expressed in Tables 1-5 and use the information and methods provided herein to interrogate or test a particular sample. For a particular interrogation of two conditions or sources, it may be desirable to select those genes which display a great deal of difference in the expression pattern between the two conditions or sources. At least a two-fold difference may be desirable, but a three-fold, five-fold or ten-fold difference may be preferred in some instances. Interrogations of the genes or proteins can be performed to yield different information.

Diagnostic Uses for the Breast Cancer Markers

As described herein, the genes and gene expression information provided in Tables 1-5 may be used as diagnostic markers for the prediction or identification of the malignant state of breast tissue. For instance, a breast tissue sample or other sample from a patient may be assayed by any of the methods known to those skilled in the art, and the expression levels from one or more genes from Tables 1-5, may be compared to the expression levels found in normal breast tissue, tissue from breast carcinoma or both. Expression profiles generated from the tissue or other samples that substantially resemble an expression profile from normal or diseased breast tissue may be used, for instance, to aid in disease diagnosis. Comparison of the expression data, as well as available sequence or other information may be done by researcher or diagnostician or may be done with the aid of a computer and databases as described herein.

For example, genes over-expressed by 3-fold or greater, as well as having the smallest p-values from a t-test, were discovered by comparing 13 normal tissue samples and 15 infiltrating ductal carcinoma tissue samples composed of mostly stage II and III tissue samples. This analysis provided a set of genes (listed in Table 1) capable of distinguishing between the 13 normal and 15 tumor samples by PCA (Principal Component Analysis). In order to evaluate the ability of the genes to distinguish between normal and tumor tissue samples, a group of 33 tissues was selected from an existing gene expression database composed of normal, benign, DCIS (ductal carcinoma in-situ), microinvasive, stage I, stage II, and stage III breast cancer samples. PCA of the 33 tissue samples indicated that the genes selected based on the smallest p-values classified 32 out of 33 tissue samples correctly, while one stage I tissue sample was misclassified as a normal sample. Accordingly, these genes can be used diagnostically to differentiate normal/benign samples from tissue samples containing intraductal or infiltrating ductal carcinoma of the breast.

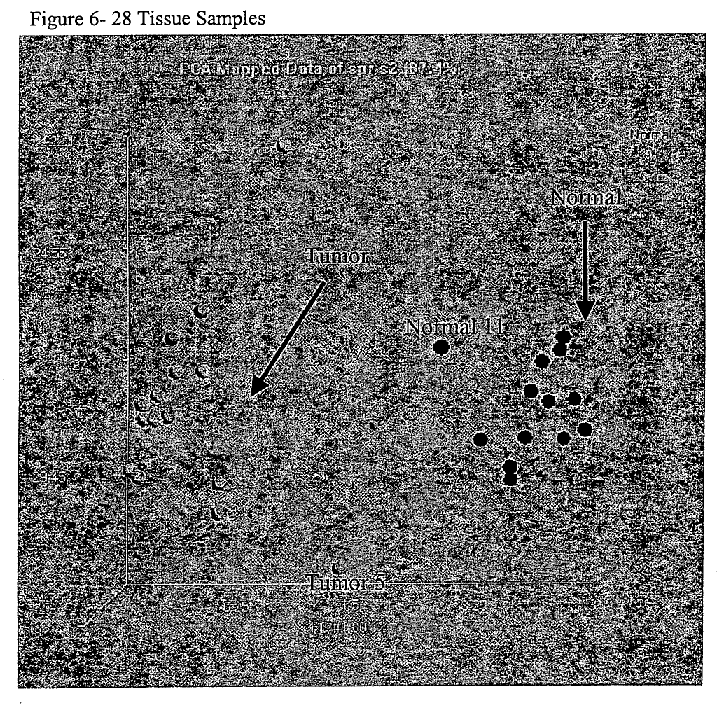

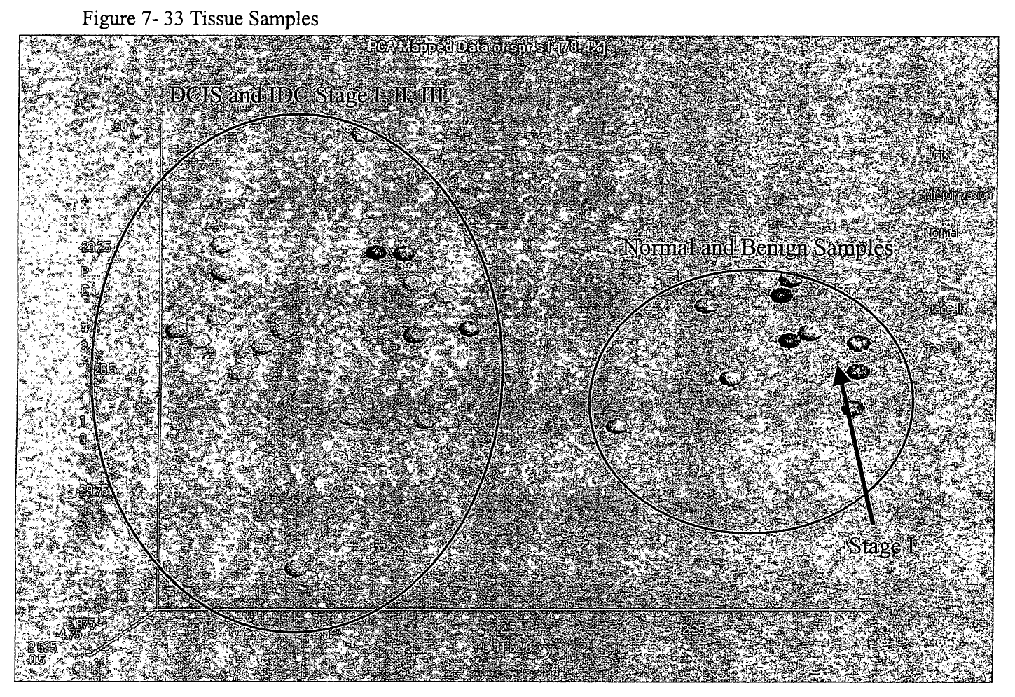

In another study, the PCA based on this group of genes indicates that these genes may be used to differentiate between the different stages of breast cancer such as normal versus DCIS or DCIS versus microinvasive tissue samples as graphically shown in FIGS. 6 and 7. The DCIS sample that contained focal microinvasions was grouped with the Stage I and II tumor samples. This group of genes may be used to determine if a DCIS sample contains microinvasions.

Use of the Breast Cancer Markers for Monitoring Disease Progression

Molecular expression markers for breast cancer can be used to confirm the type and progression of cancer made on the basis of morphological criteria. For example, normal breast tissue could be distinguished from invasive carcinoma based on the level and type of genes expressed in a tissue sample. In some situations, identifications of cell type or source is ambiguous based on classical criteria. In these situations, the molecular expression markers of the present invention are useful.

In addition, progression of ductal carcinoma in situ to microinvasive carcinoma can be monitored by following the expression patterns of the involved genes using the molecular expression markers of the present invention. Monitoring of the efficacy of certain drug regimens can also be accomplished by following the expression patterns of the molecular expression markers.

In addition to the different disease progression stages which have been shown in FIGS. 6-7, as shown in the examples below, other developmental stages can be identified using these same molecular expression markers. While the importance of these markers in development has been shown here, variations in their expression may occur at other times. For example, variation in the expression level of one or more of the marker genes identified herein may be use to distinguish benign stages of breast cancer from malignant states.

As described above, the genes and gene expression information provided in Tables 1-5 may also be used as markers for the direct monitoring of disease progression, for instance, the development of breast cancer. For instance, a breast tissue sample or other sample from a patient may be assayed by any of the methods known to those of skill in the art, and the expression levels in the sample from a gene or genes from Tables 1-5 may be compared to the expression levels found in normal breast tissue, tissue from breast cancer or both. Comparison of the expression data, as well as available sequence or other information may be done by researcher or diagnostician or may be done with the aid of a computer and databases as described herein.

For instance, methods of this invention may use the 35 gene group (profile) composed of genes expressed in myoepithelial cells and basal lamina components in Table 3. The absence of both myoepithelial cells or basement membrane components usually indicates that the intraductal carcinoma is invasive. This group of 35 genes listed in Table 3 may be used to determine if myoepithelial and/or basal lamina components are present in a tissue sample. It includes 23 genes exhibiting a fold change of 3 fold or higher and 12 genes displaying a change of less than 3 fold. This group of 23 genes was used to distinguish between normal and tumor samples for a group of 33 tissue samples. In this study, the 23 genes were able to classify 32 out of 33 samples correctly and 26 out of 28 samples used to isolate this subgroup of genes. This group of genes can be used to identify the various stages of ductal carcinoma tissues more discretely than the 50-gene set. The study also demonstrates that this group of genes can differentiate between DCIS and a cribiform type of DCIS that is more prone to microinvasion. Clinically, the ability to discern DCIS with microinvasions or phenotypes prone to microinvasions such as the cribiform type would allow subgrouping of the samples containing microinvasions as a type of patient that should be treated more aggressively than DCIS patients lacking this gene expression fingerprint. A subclass of DCIS (cribiform type) based on the gene expression fingerprint may be subgrouped as a micro invasive sample based on the gene expression pattern associated with this sample.

Use of the Breast Cancer Markers for Drug Screening

According to the present invention, potential drugs can be screened to determine if application of the drug alters the expression of one or more of the genes identified herein. This may be useful, for example, in determining whether a particular drug is effective in treating a particular patient with breast cancer. In the case where a gene's expression is affected by the potential drug such that its level of expression returns to normal, the drug is indicated in the treatment of breast cancer. Similarly, a drug which causes expression of a gene which is not normally expressed by epithelial cells in the breast, may be contra-indicated in the treatment of breast cancer.

According to the present invention, the genes identified in Tables 1-5 may also be used as markers to evaluate the effects of a candidate drug or agent on a cell, particularly a cell undergoing malignant transformation, for instance, a breast cancer cell or tissue sample. A candidate drug or agent can be screened for the ability to stimulate the transcription or expression of a given marker or markers (drug targets) or to down-regulate or inhibit the transcription or expression of a marker or markers. According to the present invention, one can also compare the specificity of a drug's effects by looking at the number of markers affected by the drug and comparing them to the number of markers affected by a different drug. A more specific drug will affect fewer transcriptional targets. Similar sets of markers identified for two drugs indicates a similarity of effects.

Assays to monitor the expression of a marker or markers as defined in Tables 1-5 may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell.

Agents that are assayed in the above methods can be randomly selected or rationally selected or designed. As used herein, an agent is said to be randomly selected when the agent is chosen randomly without considering the specific sequences involved in the association of the a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a nonrandom basis which takes into account the sequence of the target site and/or its conformation in connection with the agents action. Agents can be selected or designed by utilizing the peptide sequences that make up these sites. For example, a rationally selected peptide agent can be a peptide whose amino acid sequence is identical to or a derivative of any functional consensus site.

The agents of the present invention can be, as examples, peptides, small chemical molecules, vitamin derivatives, as well as carbohydrates, lipids, oligonucleotides and covalent and non-covalent combinations thereof. Dominant negative proteins, DNA encoding these proteins, antibodies to these proteins, peptide fragments of these proteins or mimics of these proteins may be introduced into cells to affect function. “Mimic” as used herein refers to the modification of a region or several regions of a peptide molecule to provide a structure chemically different from the parent peptide but topographically and functionally similar to the parent peptide (see Grant in Molecular Biology and Biotechnology, Meyers, ed., VCH Publishers (1995)). A skilled artisan can readily recognize that there is no limit as to the structural nature of the agents of the present invention.

Assay Formats

The genes identified as being differentially expressed in breast cancer may be used in a variety of nucleic acid detection assays to detect or quantify the expression level of a gene or multiple genes in a given sample. For example, traditional Northern blotting, nuclease protection, RT-PCR and differential display methods may be used for detecting gene expression levels.

The protein products of the genes identified herein can also be assayed to determine the amount of expression. Methods for assaying for a protein include Western blot, immunoprecipitation, radioimmunoassay. It is preferred, however, that the mRNA be assayed as an indication of expression. Methods for assaying for mRNA include Northern blots, slot blots, dot blots, and hybridization to an ordered array of oligonucleotides. Any method for specifically and quantitatively measuring a specific protein or mRNA or DNA product can be used. However, methods and assays of the invention are most efficiently designed with PCR or array or chip hybridization-based methods for detecting the expression of a large number of genes.

Any hybridization assay format may be used, including solution-based and solid support-based assay formats. A preferred solid support is a high density array also known as a DNA chip or a gene chip. One variation of the DNA chip contains hundreds of thousands of discrete microscopic channels that pass completely through it. Probe molecules are attached to the inner surface of these channels, and molecules from the samples to be tested flow through the channels, coming into close proximity with the probes for hybridization. In one assay format, gene chips containing probes to at least two genes from Tables 1-5 may be used to directly monitor or detect changes in gene expression in the treated or exposed cell as described herein. Assays of the invention may measure the expression levels of about one, two, three, five, seven, ten, 15, 20, 25, 50, 100 or more genes in the Tables.

The genes and ESTs of the present invention may be assayed in any convenient sample form. For example, samples may be assayed in the form mRNA or reverse transcribed mRNA. Samples may be cloned or not and the samples or individual genes may be amplified or not. The cloning itself does not appear to bias the representation of genes within a population. However, it may be preferable to use polyA+ RNA as a source, as it can be used with less processing steps. In some embodiments, it may be preferable to assay the protein or peptide expressed by the gene.

The sequences of the expression marker genes of Tables 1-5 are available in the public databases. Tables 1-5 provide the Accession numbers and name for each of the sequences. The sequences of the genes in GenBank are herein expressly incorporated by reference in their entirety as of the filing date of this application. (see www.ncbi.nim.nih.gov.

Additional assay formats may be used to monitor the ability of the agent to modulate the expression of a gene identified in Tables 1-5. For instance, as described above, mRNA expression may be monitored directly by hybridization of probes to the nucleic acids of the invention. Cell lines are exposed to an agent to be tested under appropriate conditions and time and total RNA or mRNA is isolated by standard procedures such those disclosed in Sambrook et al., Molecular Cloning—A Laboratory Manual, Cold Spring Harbor Laboratory Press, Cold Spring Harbor, N.Y. (1989)). In some embodiments, it may be desirable to amplify one or more of the RNA molecules isolated prior to application of the RNA to the gene chip. Using techniques well known in the art, the RNA may be reverse transcribed and amplified in the form of DNA or may be reverse transcribed into DNA and the DNA used as a template for transcription to generate recombinant RNA. Any method that results in the production of a sufficient quantity of nucleic acid to be hybridized effectively to the gene chip may be used.

In another format, cell lines that contain reporter gene fusions between the open reading frame and or the 3′ or 5′ regulatory regions of a gene in Tables 1-5 and any assayable fusion partner may be prepared. Numerous assayable fusion partners are known and readily available including the firefly luciferase gene and the gene encoding chloramphenicol acetyltransferase (Alam et al., Anal Biochem 188, 245-254 (1990)). Cell lines containing the reporter gene fusions are then exposed to the agent to be tested under appropriate conditions and time. Differential expression of the reporter gene between samples exposed to the agent and control samples identifies agents which modulate the expression of the nucleic acid.

In another assay format, cells or cell lines are first identified which express one or more of the gene products of the invention physiologically. Cells and/or cell lines so identified would preferably comprise the necessary cellular machinery to ensure that the transcriptional and/or translational apparatus of the cells would faithfully mimic the response of normal or cancerous breast tissue to an exogenous agent. Such machinery would likely include appropriate surface transduction mechanisms and/or cytosolic factors. Such cell lines may be, but are not required to be, derived from breast tissue. The cells and/or cell lines may then be contacted with an agent and the expression of one or more of the genes of interest may then be assayed. The genes may be assayed at the mRNA level and/or at the protein level.

In some embodiments, such cells or cell lines may be transduced or transfected with an expression vehicle (e.g., a plasmid or viral vector) containing an expression construct comprising an operable 5′-promoter containing end of a gene of interest identified in Tables 1-5 fused to one or more nucleic acid sequences encoding one or more antigenic fragments. The construct may comprise all or a portion of the coding sequence of the gene of interest which may be positioned 5′- or 3′- to a sequence encoding an antigenic fragment. The coding sequence of the gene of interest may be translated or un-translated after transcription of the gene fusion. At least one antigenic fragment may be translated. The antigenic fragments are selected so that the fragments are under the transcriptional control of the promoter of the gene of interest and are expressed in a fashion substantially similar to the expression pattern of the gene of interest. The antigenic fragments may be expressed as polypeptides whose molecular weight can be distinguished from the naturally occurring polypeptides. In some embodiments, gene products of the invention may further comprise an immunologically distinct tag. Such a process is well known in the art (see Sambrook et al., supra).

Cells or cell lines transduced or transfected as outlined above are then contacted with agents under appropriate conditions; for example, the agent comprises a pharmaceutically acceptable excipient and is contacted with cells comprised in an aqueous physiological buffer such as phosphate buffered saline (PBS) at physiological pH, Eagles balanced salt solution (BSS) at physiological pH, PBS or BSS comprising serum or conditioned media comprising PBS or BSS and serum incubated at 37° C. Said conditions may be modulated as deemed necessary by one of skill in the art. Subsequent to contacting the cells with the agent, said cells will be disrupted and the polypeptides of the lysate are fractionated such that a polypeptide fraction is pooled and contacted with an antibody to be further processed by immunological assay (e.g., ELISA, immunoprecipitation or Western blot). The pool of proteins isolated from the “agent-contacted” sample will be compared with a control sample where only the excipient is contacted with the cells and an increase or decrease in the immunologically generated signal from the “agent-contacted” sample compared to the control will be used to distinguish the effectiveness of the agent.

Another embodiment of the present invention provides methods for identifying agents that modulate the levels, concentration or at least one activity of a protein(s) encoded by the genes in Tables 1-5. Such methods or assays may utilize any means of monitoring or detecting the desired activity.

In one format, the relative amounts of a protein of the invention produced in a cell population that has been exposed to the agent to be tested may be compared to the amount produced in an un-exposed control cell population. In this format, probes such as specific antibodies are used to monitor the differential expression of the protein in the different cell populations. Cell lines or populations are exposed to the agent to be tested under appropriate conditions and time. Cellular lysates may be prepared from the exposed cell line or population and a control, unexposed cell line or population. The cellular lysates are then analyzed with the probe, such as a specific antibody.

Probe Design

Probes based on the sequences of the genes described herein may be prepared by any commonly available method. Oligonucleotide probes for assaying the tissue or cell sample are preferably of sufficient length to specifically hybridize only to appropriate, complementary genes or transcripts. Typically the oligonucleotide probes will be at least 10, 12, 14, 16, 18, 20 or 25 nucleotides in length. In some cases longer probes of at least 30, 40, or 50 nucleotides will be desirable.

One of skill in the art will appreciate that an enormous number of array designs are suitable for the practice of this invention. The high density array will typically include a number of probes that specifically hybridize to the sequences of interest. See WO 99/32660 for methods of producing probes for a given gene or genes. In addition, in a preferred embodiment, the array will include one or more control probes.

High density array chips of the invention include “test probes.” Test probes may be oligonucleotides that range from about 5 to about 500 or about 5 to about 50 nucleotides, more preferably from about 10 to about 40 nucleotides and most preferably from about 15 to about 40 nucleotides in length. In other particularly preferred embodiments, the probes are about 20 or 25 nucleotides in length. In another preferred embodiment, test probes are double or single strand DNA sequences. DNA sequences may be isolated or cloned from natural sources or amplified from natural sources using natural nucleic acid as templates. These probes have sequences complementary to particular subsequences of the genes whose expression they are designed to detect. Thus, the test probes are capable of specifically hybridizing to the target nucleic acid they are to detect.

In addition to test probes that bind the target nucleic acid(s) of interest, the high density array can contain a number of control probes. The control probes fall into three categories referred to herein as (1) normalization controls; (2) expression level controls; and (3) mismatch controls.

Normalization controls are oligonucleotide or other nucleic acid probes that are complementary to labeled reference oligonucleotides or other nucleic acid sequences that are added to the nucleic acid sample. The signals obtained from the normalization controls after hybridization provide a control for variations in hybridization conditions, label intensity, “reading” efficiency and other factors that may cause the signal of a perfect hybridization to vary between arrays. In a preferred embodiment, signals (e.g., fluorescence intensity) read from all other probes in the array are divided by the signal (e.g., fluorescence intensity) from the control probes thereby normalizing the measurements.

Virtually any probe may serve as a normalization control. However, it is recognized that hybridization efficiency varies with base composition and probe length. Preferred normalization probes are selected to reflect the average length of the other probes present in the array, however, they can be selected to cover a range of lengths. The normalization control(s) can also be selected to reflect the (average) base composition of the other probes in the array, however in a preferred embodiment, only one or a few probes are used and they are selected such that they hybridize well (i.e., no secondary structure) and do not match any target-specific probes.

Expression level controls are probes that hybridize specifically with constitutively expressed genes in the biological sample. Virtually any constitutively expressed gene provides a suitable target for expression level controls. Typical expression level control probes have sequences complementary to subsequences of constitutively expressed “housekeeping genes” including, but not limited to the β-actin gene, the transferrin receptor gene, the GAPDH gene, and the like.

Mismatch controls may also be provided for the probes to the target genes, for expression level controls or for normalization controls. Mismatch controls are oligonucleotide probes or other nucleic acid probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent). Preferred mismatch probes contain a central mismatch. Thus, for example, where a probe is a twenty-mer, a corresponding mismatch probe may have the identical sequence except for a single base mismatch (e.g., substituting a G, a C or a T for an A) at any of positions 6 through 14 (the central mismatch).

Mismatch probes thus provide a control for non-specific binding or cross hybridization to a nucleic acid in the sample other than the target to which the probe is directed. Mismatch probes also indicate whether a hybridization is specific or not. For example, if the target is present the perfect match probes should be consistently brighter than the mismatch probes. In addition, if all central mismatches are present, the mismatch probes can be used to detect a mutation. The difference in intensity between the perfect match and the mismatch probe (I(PM)-I(MM)) provides a good measure of the concentration of the hybridized material.

Nucleic Acid Samples

As is apparent to one of ordinary skill in the art, nucleic acid samples used in the methods and assays of the invention may be prepared by any available method or process. Methods of isolating total mRNA are also well known to those of skill in the art. For example, methods of isolation and purification of nucleic acids are described in detail in Chapter 3 of Laboratory Techniques in Biochemistry and Molecular Biology, Vol. 24, Hybridization With Nucleic Acid Probes: Theory and Nucleic Acid Probes, P. Tijssen, ed., Elsevier Press, New York (1993). Such samples include RNA samples, but also include cDNA synthesized from a mRNA sample isolated from a cell or tissue of interest. Such samples also include DNA amplified from the cDNA, and an RNA transcribed from the amplified DNA. One of skill in the art would appreciate that it may be desirable to inhibit or destroy RNase present in homogenates before homogenates can be used.

Biological samples may be of any biological tissue or fluid or cells from any organism as well as cells raised in vitro, such as cell lines and tissue culture cells. Frequently the sample will be a “clinical sample” which is a sample derived from a patient. Typical clinical samples include, but are not limited to, breast tissue biopsy, sputum, blood, blood-cells (e.g., white cells), tissue or fine needle biopsy samples, urine, peritoneal fluid, and pleural fluid, or cells therefrom.

Biological samples may also include sections of tissues, such as frozen sections or formalin fixed sections taken for histological purposes.

Solid Supports

Solid supports containing oligonucleotide probes for differentially expressed genes can be any solid or semisolid support material known to those skilled in the art. Suitable examples include, but are not limited to, membranes, filters, tissue culture dishes, polyvinyl chloride dishes, beads, test strips, silicon or glass based chips and the like. Suitable glass wafers and hybridization methods are widely available, for example, those disclosed by Beattie (WO 95/11755). Any solid surface to which oligonucleotides can be bound, either directly or indirectly, either covalently or non-covalently, can be used. In some embodiments, it may be desirable to attach some oligonucleotides covalently and others non-covalently to the same solid support.

A preferred solid support is a high density array or DNA chip. These contain a particular oligonucleotide probe in a predetermined location on the array. Each predetermined location may contain more than one molecule of the probe, but each molecule within the predetermined location has an identical sequence. Such predetermined locations are termed features. There may be, for example, from 2, 10, 100, 1000 to 10,000, 100,000 or 400,000 of such features on a single solid support. The solid support, or the area within which the probes are attached may be on the order of a square centimeter.

Oligonucleotide probe arrays for expression monitoring can be made and used according to any techniques known in the art (see for example, Lockhart et al., Nat Biotechnol 14, 1675-1680 (1996); McGall et al., Proc Nat Acad Sci USA 93, 13555-13460 (1996)). Such probe arrays may contain at least two or more oligonucleotides that are complementary to or hybridize to two or more of the genes described herein. Such arrays my also contain oligonucleotides that are complementary or hybridize to at least 3, 4, 5, 6, 7, 8, 9, 10, 20, 30, 50, 70 or more the genes described herein.

Methods of forming high density arrays of oligonucleotides with a minimal number of synthetic steps are known. The oligonucleotide analogue array can be synthesized on a solid substrate by a variety of methods, including, but not limited to, light-directed chemical coupling, and mechanically directed coupling (see Pirrung et al., (1992) U.S. Pat. No. 5,143,854; Fodor et al., (1998) U.S. Pat. No. 5,800,992; Chee et al., (1998) U.S. Pat. No. 5,837,832).

In brief, the light-directed combinatorial synthesis of oligonucleotide arrays on a glass surface proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′ photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

In addition to the foregoing, additional methods which can be used to generate an array of oligonucleotides on a single substrate are described in Fodor et al., WO 93/09668. High density nucleic acid arrays can also be fabricated by depositing pre-made or natural nucleic acids in predetermined positions. Synthesized or natural nucleic acids are deposited on specific locations of a substrate by light directed targeting and oligonucleotide directed targeting. Another embodiment uses a dispenser that moves from region to region to deposit nucleic acids in specific spots.

Hybridization

Nucleic acid hybridization simply involves contacting a probe and target nucleic acid under conditions where the probe and its complementary target can form stable hybrid duplexes through complementary base pairing (see Lockhart et al., WO 99/32660). The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA-DNA, RNA-RNA or RNA-DNA) will form even where the annealed sequences are not perfectly complementary. Thus, specificity of hybridization is reduced at lower stringency. Conversely, at higher stringency (e.g., higher temperature or lower salt) successful hybridization requires fewer mismatches. One of skill in the art will appreciate that hybridization conditions may be selected to provide any degree of stringency. In a preferred embodiment, hybridization is performed at low stringency, in this case in 6×SSPE-T at 37° C. (0.005% Triton x-100) to ensure hybridization and then subsequent washes are performed at higher stringency (e.g., 1×SSPE-T at 37° C.) to eliminate mismatched hybrid duplexes. Successive washes may be performed at increasingly higher stringency (e.g., down to as low as 0.25×SSPET at 37° C. to 50° C.) until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

Signal Detection

The hybridized nucleic acids are typically detected by detecting one or more labels attached to the sample nucleic acids. The labels may be incorporated by any of a number of means well known to those of skill in the art (see Lockhart et al., WO 99/32660).

Databases

The present invention includes relational databases containing sequence information, for instance for one or more of the genes of Tables 1-5, as well as gene expression information in various breast tissue samples. Databases may also contain information associated with a given sequence or tissue sample such as descriptive information about the gene associated with the sequence information, descriptive information concerning the clinical status of the tissue sample, or information concerning the patient from which the sample was derived. The database may be designed to include different parts, for instance a sequence database and a gene expression database. Methods for the configuration and construction of such databases are widely available, for instance, see Akerblom et al., (1999) U.S. Pat. No. 5,953,727, which is specifically incorporated herein by reference in its entirety.

The databases of the invention may be linked to an outside or external database. In a preferred embodiment, as described in Tables 1-5, the external database is GenBank and the associated databases maintained by the National Center for Biotechnology Information (NCBI).

Any appropriate computer platform may be used to perform the necessary comparisons between sequence information, gene expression information and any other information in the database or provided as an input. For example, a large number of computer workstations are available from a variety of manufacturers, such has those available from Silicon Graphics. Client-server environments, database servers and networks are also widely available and appropriate platforms for the databases of the invention.

The databases of the invention may be used to produce, among other things, electronic Northern blots (E-Northerns) to allow the user to determine the cell type or tissue in which a given gene is expressed and to allow determination of the abundance or expression level of a given gene in a particular tissue or cell. The E-northern analysis can be used as a tool to discover tissue specific candidate therapeutic targets that are not over-expressed in tissues such as the liver, kidney, or heart. These tissue types often lead to detrimental side effects once drugs are developed and a first-pass screen to eliminate these targets early in the target discovery and validation process would be beneficial.

The databases of the invention may also be used to present information identifying the expression level in a tissue or cell of a set of genes comprising at least one gene in Tables 1-5 comprising the step of comparing the expression level of at least one gene in Tables 1-5 in the tissue to the level of expression of the gene in the database. Such methods may be used to predict the physiological state of a given tissue by comparing the level of expression of a gene or genes in Tables 1-5 from a sample to the expression levels found in tissue from normal breast tissue, tissue from breast carcinoma or both. Such methods may also be used in the drug or agent screening assays as described herein.

Kits

The invention further includes kits combining, in different combinations, high-density oligonucleotide arrays, reagents for use with the arrays, signal detection and array-processing instruments, gene expression databases and analysis and database management software described above. The kits may be used, for example, to monitor the progression of breast cancer, to identify genes that show promise as new drug targets and to screen known and newly designed drugs as discussed above.

The databases packaged with the kits are a typically a compilation of expression patterns from human breast cancer tissue or cell lines and for gene and gene fragments as described herein (corresponding to the genes of Tables 1-5). In particular, the database software and packaged information include the expression results of Tables 1-5 that can be used to predict the cancerous state of a tissue sample by comparing the expression levels of the genes in the tissue or cell sample to the expression levels presented in Tables 1-5.

The kits may used in the pharmaceutical industry, where the need for early drug testing is strong due to the high costs associated with drug development, but where bioinformatics, in particular gene expression informatics, is still lacking. These kits will reduce the costs, time and risks associated with traditional new drug screening using cell cultures and laboratory animals. The results of large-scale drug screening of pre-grouped patient populations, pharmacogenomics testing, can also be applied to select drugs with greater efficacy and fewer side-effects. The kits may also be used by smaller biotechnology companies and research institutes who do not have the facilities for performing such large-scale testing themselves.

Databases and software designed for use with use with microarrays is discussed in Balaban et al., (2001) U.S. Pat. No. 6,229,911, a computer-implemented method for managing information, stored as indexed tables, collected from small or large numbers of microarrays, and U.S. Pat. No. 6,185,561, a computer-based method with data mining capability for collecting gene expression level data, adding additional attributes and reformatting the data to produce answers to various queries. Chee et al., (1999) U.S. Pat. No. 5,974,164, disclose a software-based method for identifying mutations in a nucleic acid sequence based on differences in probe fluorescence intensities between wild type and mutant sequences that hybridize to reference sequences. The object of the method is to predict regions or positions of mutation.

Without further description, it is believed that one of ordinary skill in the art can, using the preceding description and the following illustrative examples, make and utilize the compounds of the present invention and practice the claimed methods. The preceding working examples therefore, are illustrative only and should not be construed as limiting in any way the scope of the invention.

### EXAMPLES

### Example 1

### Preparation of Breast Cancer Profiles

Tissue Sample Acquisition and Preparation

The patient tissue samples were derived from female patients; the average age for the normal and tumor samples was 39 and 52 years respectively. They stem from three different ethnic origins (Caucasian, African-American, and Asian). Furthermore, all tissue samples from Infiltrating Ductal Carcinoa (IDC) patient samples were studied for cancer-related expression, as 85% of the breast cancer patients were afflicted with this form of the disease. The samples are composed of normal, benign, DCIS (ductal carcinoma in-situ), microinvasive, stage I, stage II, and stage III breast cancer samples.

Histological analysis of each of the tissue samples Was performed and samples were segregated into either normal or malignant categories. The normal tissue samples were acquired from neighboring tissue of patients suffering from one of the following disorders: macromastia, mild fibrosis, infiltrating lobular carcinoma, or infiltrating ducal carcinoma, however; each tissue was diagnosed as normal by histological analysis.

With minor modifications, the sample preparation protocol followed the Affymetrix GeneChip Expression Analysis Manual. Frozen tissue was first ground to powder using the Spex Certiprep 6800 Freezer Mill. Total RNA was then extracted using Trizol (Life Technologies). The total RNA yield for each sample (average tissue weight of 300 mg) was 200-500 μg. Next, mRNA was isolated using the Oligotex mRNA Midi kit (Qiagen). Since the mRNA was eluted in a final volume of 400 μl, an ethanol precipitation step was required to bring the concentration to 1 μg/μl. Using 1-5 μg of mRNA, double stranded cDNA was created using the SuperScript Choice system (Gibco-BRL). First strand cDNA synthesis was primed with a T7-(dT₂₄) oligonucleotide. The cDNA was then phenol-chloroform extracted and ethanol precipitated to a final concentration of 1 μg/μl.

From 2 μg of cDNA, cRNA was synthesized according to standard procedures. To biotin label the cRNA, nucleotides Bio-11-CTP and Bio-16-UTP (Enzo Diagnostics) were added to the reaction. After a 37° C. incubation for six hours, the labeled cRNA was cleaned up according to the Rneasy Mini kit protocol (Qiagen). The cRNA was then fragmented (5×fragmentation buffer: 200 mM Tris-Acetate (pH 8.1), 500 mM KOAc, 150 mM MgOAc) for thirty-five minutes at 94° C.

55 μg of fragmented cRNA was hybridized on the human and the Human Genome U95 set of arrays for twenty-four hours at 60 rpm in a 45° C. hybridization oven. The chips were washed and stained with Streptavidin Phycoerythrin (SAPE) (Molecular Probes) in Affymetrix fluidics stations. To amplify staining, SAPE solution was added twice with an anti-streptavidin biotinylated antibody (Vector Laboratories) staining step in between. Hybridization to the probe arrays was detected by fluorometric scanning (Hewlett Packard Gene Array Scanner). Following hybridization and scanning, the microarray images were analyzed for quality control, looking for major chip defects or abnormalities in hybridization signal. After all chips passed QC, the data was analyzed using Affymetrix GeneChip software (v3.0), and Experimental Data Mining Tool (EDMT) software (v1.0).

Gene Expression Analysis

All samples were prepared as described and hybridized onto the Affymetrix Human Genome U95 array. Each chip contains 16-20 oligonucleotide probe pairs per gene or cDNA clone. These probe pairs include perfectly matched sets and mismatched sets, both of which are necessary for the calculation of the average difference. The average difference is a measure of the intensity difference for each probe pair, calculated by subtracting the intensity of the mismatch from the intensity of the perfect match. This takes into consideration variability in hybridization among probe pairs and other hybridization artifacts that could affect the fluorescence intensities. Using the average difference value that has been calculated, an absolute call for each gene or EST is made.

The absolute call of present, absent or marginal is used to generate a Gene Signature, a tool used to identify those genes that are commonly present or commonly absent in a given sample set, according to the absolute call. For each set of samples, a median average difference was calculated using the average differences of each individual sample within the set. The median average difference typically must be greater than 20 to assure that the expression level is at least two standard deviations above the background noise of the hybridization. For the purposes of this study, only the genes and gene fragments with a median average difference greater than 20 were further studied in detail.

The Gene Signature for one set of samples is compared to the Gene Signature of another set of samples to determine the Gene Signature Differential. This comparison identifies the genes that are consistently present in one set of samples and consistently absent in the second set of samples.

The Gene Signature Curve is a graphic view of the number of genes consistently present in a given set of samples as the sample size increases, taking into account the genes commonly expressed among a particular set of samples, and discounting those genes whose expression is variable among those samples. The curve is also indicative of the number of samples necessary to generate an accurate Gene Signature. As the sample number increases, the number of genes common to the sample set decreases. The curve is generated using the positive Gene Signatures of the samples in question, determined by adding one sample at a time to the Gene Signature, beginning with the sample with the smallest number of present genes and adding samples in ascending order. The curve displays the sample size required for the most consistency and the least amount of expression variability from sample to sample. The point where this curve begins to level off represents the minimum number of samples required for the Gene Signature. Graphed on the x-axis is the number of samples in the set, and on the y-axis is the number of genes in the positive Gene Signature. As a general rule, the acceptable percent of variability in the number of positive genes between two sample sets should be less than 5%.

Fold Change Analysis

The data was first filtered to exclude all genes that showed no expression in any of the samples. The ratio (tumor/normal) was calculated by comparing the mean expression value for each gene in the breast cancer sample set against the mean expression value of that gene in the normal breast sample set. For Table 2, genes were included in the analysis if they had a fold change ≧3 in either direction, and a p-value <0.05 as determined by a two-tail unequal variance t-test. Out of the ˜60,000 genes surveyed by the Human Genome U95 set, 802 genes were present in the overall fold change analysis

Expression Profiles of Genes Differentially Expressed in Breast Cancer

Using the above described methods, genes that were predominantly over-expressed in breast cancer, or predominantly under-expressed in breast cancer were identified. Genes with consistent differential expression patterns provide potential targets for broad range diagnostics and therapeutics. For simplicity, applicants examined known genes by hierarchical cluster analysis developed by Eisen and colleagues to determine if functionally related genes would cluster together (see Eisen, et al. Proc Natl Acad Sci USA 95, 14863-14868 (1998)).

Table 2 lists the genes determined to be differentially expressed in cancerous breast tissues compared to normal breast tissue, with the fold change value for each gene. These genes or subsets of these genes comprise an overall breast cancer gene expression profile.

The well-characterized proliferation marker for breast cancer KI-67 had an average-fold change value of 2.8, which was calculated from 15 IDC tissue samples analyzed (see Gerdes, Semin Cancer Biol 1, 199-206 (1990)). As the fold change was below the present 3 fold criteria, the fold change value was not presented in Table 2. Some genes previously shown to be over or under expressed in breast cancer as indicated from the literature such as cytokeratins 5, 14, 15, 17, maspin, MMP 9 and 11, fibronectin, and pituitary tumor transforming 1, etc. are displayed in Table 2 as well as some genes such as p57(kip2), p63/p51/KET, mitosin, and pCDC55 whose expression levels were not previously known to vary in breast cancer.

The pituitary-tumor transforming 1 gene has been shown to produce in vitro and in vivo tumor-inducing activity (see Zhang et al. Mol Endocrinol 13, 156-66 (1999). In a recent publication, pituitary-tumor transforming 1 has been shown to be over-expressed in mammary adenocarcinomas (see Saez et al. Oncogene 18, 5473-6 (1999)). Also, another study discovered that all 48 colon carcinomas examined over-expressed PTTG1 as compared to normal colorectal tissue, and invasion of the surrounding tissue was associated with higher PTTG1 expression levels (see Heaney et al. Expression of pituitary-tumour transforming gene in colorectal tumours [see comments] Lancet 355, 716-9 (2000)).

Genes listed in Table 2, not reported in the literature to be over-expressed in human breast cancer tissues, include RAD2, FLS353, CKS2, cyclin-selective ubiquitin carrier protein E2-C, ZWINT, Lamin B 1 and H2A.X. Although FLS353 has been recently found to be over-expressed in colorectal cancer (see Hufton et al. FEBS Lett 463, 77-82 (1999)), differential expression of FLS353 in breast tumor cells had not been previously demonstrated.

Cyclin-ubiquitin carrier protein E2-C is another gene over-expressed in breast cancer, which was discovered in this study. Previous studies have shown that when a dominant-negative form of the protein is over-expressed, the mammalian cells arrested in M phase and evidence was provided indicating that this mutant form of cyclin-ubiquitin carrier protein E2-C blocked the destruction of both cyclin A and B (see Townsley et al., Proc Natl Acad Sci USA 94, 2362-7 (1997)).

The expression levels of the genes in Tables 4 and 5 are associated with various stages of infiltrating ductal carcinoma (Table 4) or infiltrating lobular carcinoma (Table 5). The Tables present the fold change value of expression in the particular disease state compared to normal breast tissue. The genes in these tables may be used alone, or in combination with those listed in Tables 1-3 in the methods, compositions, databases and computer systems of the invention.

### Example 2

### Diagnostic Subset of Breast Cancer Associated Genes

Table 1 lists the members of a diagnostic subset of genes selected by p-value. This group of genes can be used to differentiate between normal/benign and breast tumor tissue samples including two DCIS samples. Assays using these genes are capable of distinguishing between normal and tumor samples with near 100% efficiency (see FIG. 6). Only 1 of the 33 samples shown was misclassified as a normal sample based on the gene expression profile when this set of genes was used to analyze the 33 sample set (see FIG. 7).

FIGS. 6 and 7 are three-dimensional plots displaying the relationship of variance derived from gene expression data obtained from patient samples. In FIG. 6, normal tissue samples are displayed as darker spheres and the infiltrating ductal carcinoma tissue samples are the lighter spheres. The x-axis represents the first principal component that contains the greatest variance in data of 80%. The y-axis represents the second principal component of 4%. The z-axis represents the third principal component of 3%. FIG. 7 displays the results obtained from a separate 33 sample set which is composed of new samples that have no relation to the 28 sample set utilized to discover the gene set of Table 1. Again, the x, y, and z-axes represent the first (63%), second (10%), and third principal components (6%), respectively.

The gene set of Table 1 can thus be used to distinguish normal from cancerous breast tissue.

### Example 3

### Myoepithelial and Luminal Cell Marker Genes Examined on a Global Scale

Previous studies have indicated that myoepithelial cells express both epithelial and smooth muscle gene expression markers while luminal epithelial cells fail to express these genes (see Lazard et al., Proc Natl Acad Sci USA 90, 999-1003 (1993)). Cluster analysis identified a group 35 fragments representing 31 genes into one highly correlative cluster and the combination of genes and ESTs are listed in Table 3.

Previous studies have indicated that calponin and myosin heavy chain are expressed in smooth muscle cells and myoepithelial cells while luminal epithelium lack the expression of these genes. Furthermore, the proteins are usually not expressed in invasive ductal carcinoma of the breast (Lazard, et al., supra). Both calponin (fold change −11) and myosin heavy chain (fold change −10.8) were under-expressed in IDC. As indicated in Table 3, other genes associated with smooth muscle that were under-expressed such as smooth muscle gamma-actin, myosin light chain kinase, myosin, heavy polypeptide 11, and Leiomodin 1 and both mysoin polypeptide 11 and leiomodin 1 have not been previously reported to be under-expressed in breast cancer as compared to normal tissue samples.

The expression pattern represented in this particular cluster indicates that a preponderance of tissue samples diagnosed as infiltrating ductal carcinoma exhibit a luminal phenotype while myoepithelial cells were absent. More evidence to support this finding includes the under-expression of cytokeratins 5, 14, 15, and 17 in the tumor samples as shown in Table 3. Normal myoepithelial cells express cytokeratins 5, 14, 15, and 17 and breast carcinoma cells do not (Trask et al. Proc Natl Acad Sci USA 87, 2319-2323 (1990)). A previous study has indicated that myoepithelial cells are present in normal, benign lesions, grade I infiltrating ductal carcinoma but are absent in carcinomas of grades II and III (Gusterson et al. Cancer Res 42, 4763-4770 (1982)).

In addition, components of the basal lamina such as laminin were under-expressed in the infiltrating ductal carcinoma relative to normal tissue samples (Table 3). Both laminin S B3 and laminin-related protein were under-expressed as indicated in Table 3. It has been reported that myoepithelial and basal lamina markers are useful in differentiating microinvasive from ductal carcinomas of the breast (Damiani et al. Virchows Arch 434, 227-234 (1999)).

The set of 35 fragments representing 31 genes as shown in Table 3 could distinguish between intraductal carcinoma and microinvasive DCIS tissue samples based on the disappearance of genes expressed in either basal lamina or myoepithelial cells. There is evidence in the literature that the collapse of the basement membrane as well as the disappearance of an intact myoepithelial cell layer occurs during the invasion process. A multi-gene screen utilizing either of these sets of genes can be used to differentiate between benign and invasive breast neoplasm based on the gene expression fingerprint elucidated in this study.

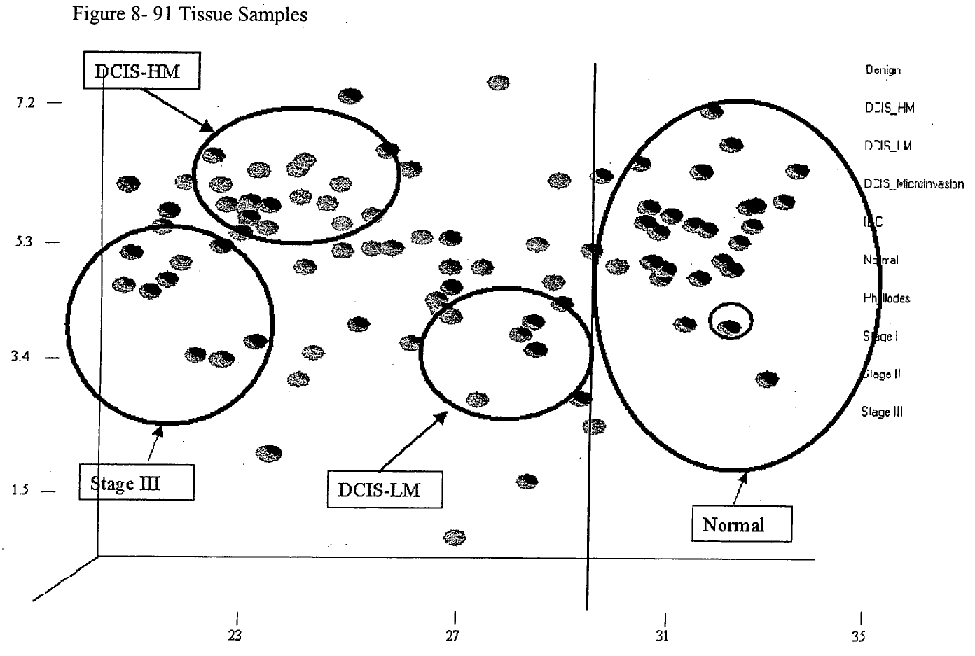

FIG. 8 shows the results of PCA of the 91 sample set with all 35 fragments (representing 31 genes and ESTs) in Table 3. These results demonstrate that PCA using the genes in Table 3 is able to distinguish between non-invasive and invasive breast tissue samples. FIG. 8 provides evidence that this group of genes is diagnostically useful for differentiating DCIS samples that are intraductal (non-invasive) from those containing microinvasion. As shown in FIG. 8, this group of genes and ESTs is capable of differentiating between two subtypes of DCIS and may constitute a set that is a more sensitive predictor of a microinvasion phenotype.

### Example 4

### Discovery of Breast Tissue Specific Genes in IDC

Electronic northern (E-northern) analysis determines if a gene of interest is present in a tissue from a database of gene expression information, and if it is present, then at what levels. Expression levels were determined using a GeneChip set that evaluated the expression levels of 60,000 genes in each type of tissue from 28 different normal human tissues. Similar to multi-tissue northern blot analysis, E-northern analysis quickly determines if a gene of interest is expressed in a particular tissue type and also at what level. E-northern analysis of multiple tissue samples can be evaluated and the determination of exactly how many samples of a particular group that express the gene of interest is tabulated and statistical analysis can be implemented. Multiple samples from the same tissue are not available at this time using conventional multi-tissue northern blot analysis.

The E-northern analysis can be used as a tool to discover tissue specific candidate therapeutic targets that are not over-expressed in tissues such as the liver, kidney, or heart. These tissue types often lead to detrimental side effects once drugs are developed and a first-pass screen to eliminate these targets early in the target discovery and validation process would be beneficial. Furthermore, different tissues have very unique gene expression profiles related to parameters such as proliferation, differentiation, or cell types contained in the tissue that can provide interesting clues into the biological roles of the ESTs.

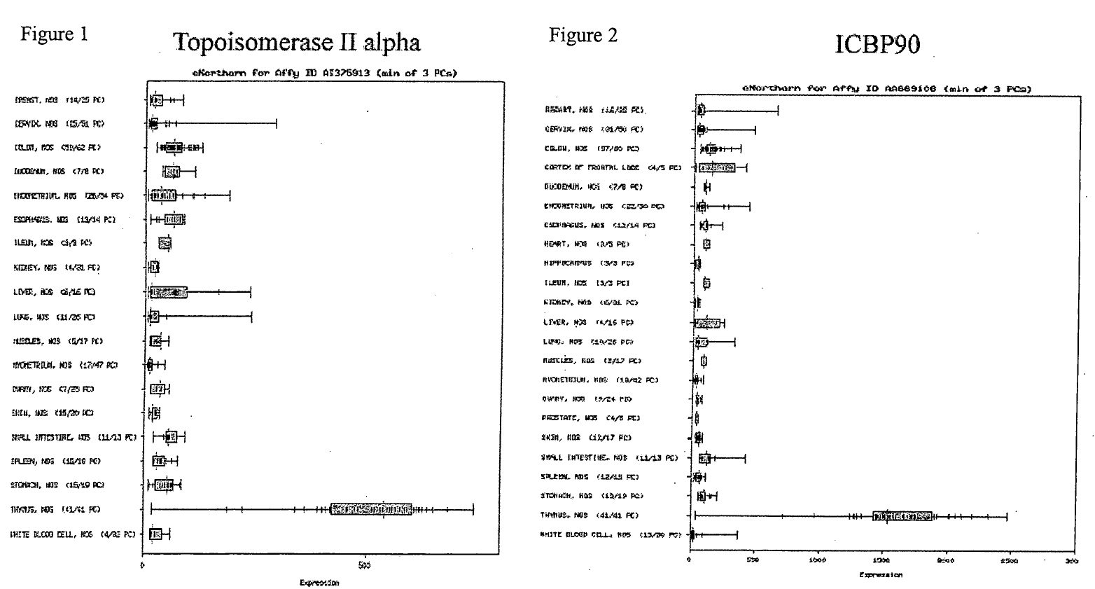

E-northern analysis was performed for many of the genes clustered in Table 2. Analysis of the E-northerns revealed that most of the genes were expressed at elevated levels in the thymus. There is high rate of mitosis present in the thymus during T-lymphocyte maturation and many proliferation-associated genes are expressed at elevated levels such as CDC2, cyclin B1, and topoisomerase II alpha. FIG. 1 displays the E-northern analysis for topoisomerase II alpha indicating elevated levels of expression in the thymus as compare to the other tissue types detected. FIG. 2 shows the results of an E-Northern analysis of transcription factor ICBP90, implicated to be involved with topoisomearse II alpha expression. ICBP90 was also expressed at high levels relative to the other tissue types in the thymus (FIG. 2). A study by Hopfner et al. indicated that adult thymus and fetal thymus contained the highest levels of ICBP90 using a 50-tissue RNA dot blot protocol (Hopfner et al. Cancer Res 60, 121-128 (2000)). Most of the genes contained in this cluster contained the highest levels of expression in the thymus.

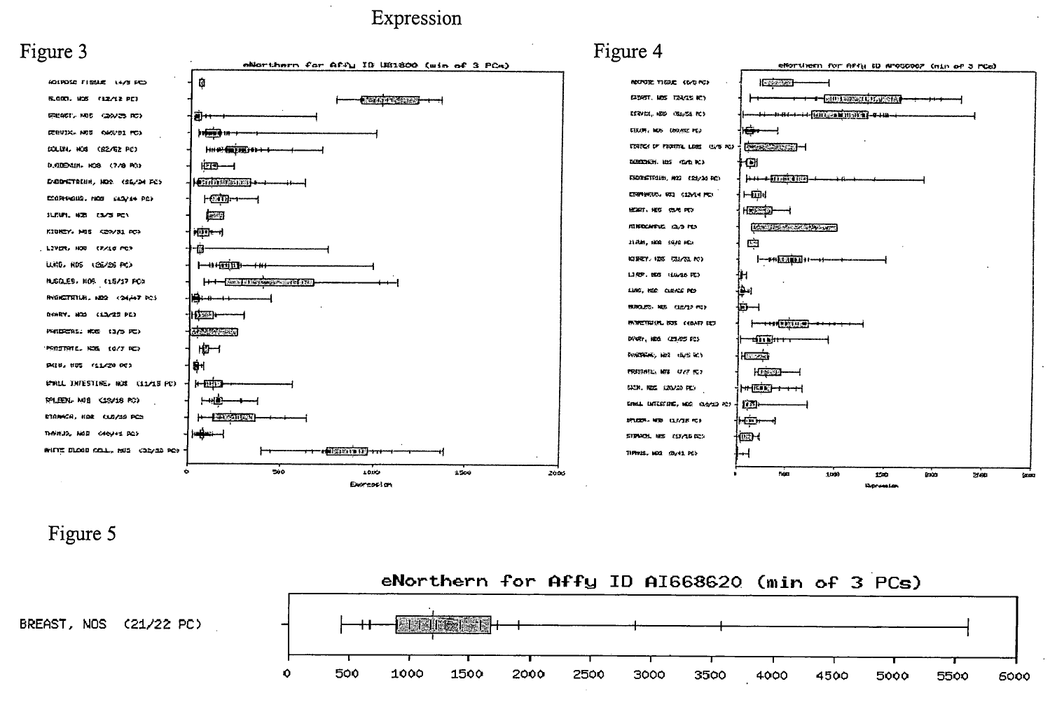

FIG. 3 shows the results of an E-Northern analysis of the monocarboxylate transporter 4 (MCT4; formerly known as MCT3) which was grouped with genes associated with proliferation. MCT4 is most evident in cells with a high glycolytic rate such as muscle, white blood cells, and tumor cells (Halestrap et al., Biochem J 343 (Pt 2), 281-299 (1999)). A group of multi-tissue northern blots from a recent publication indicate that MCT4 is expressed at high levels in leukocytes but also other tissue types as well (Price et al., Biochem J 329, 321-328 (1998)). Furthermore, electronic-northern analysis indicated high levels of MCT4 were expressed in blood and white blood cells (FIG. 3).

A previously uncharacterized gene only expressed in breast tissue was identified from this study and an E-Northern analysis of the expression pattern of this gene is shown in FIG. 4. The distribution pattern of the expression of the gene shows it be used as a marker for breast cancer. The E-northern analysis only displays tissues where the gene of interest is present at detectable levels and breast tissue was the only tissue that this particular gene was under-expressed by −4.2 fold in IDC making it particularly useful as a diagnostic marker.

Another gene that may be used as a diagnostic marker that was not present in a particular cluster is the secreted frizzled-related protein 1. This gene was under-expressed in IDC by −17.7 fold, and the E-northern analysis shown in FIG. 5 indicates that it was expressed at greatest levels in breast tissue as well as in the cervix. Using the combination of clustering, fold-change analysis, and E-northern analysis on microarray data one skilled in the art can readily select additional therapeutic and diagnostic markers.

Although the present invention has been described in detail with reference to examples above, it is understood that various modifications can be made without departing from the spirit of the invention. Accordingly, the invention is limited only by the following claims. All cited patents and publications referred to in this application are herein incorporated by reference in their entirety.

LENGTHY TABLE REFERENCED HERE
US20070015148A1-20070118-T00001
Please refer to the end of the specification for access instructions.

LENGTHY TABLE REFERENCED HERE
US20070015148A1-20070118-T00002
Please refer to the end of the specification for access instructions.

LENGTHY TABLE REFERENCED HERE
US20070015148A1-20070118-T00003
Please refer to the end of the specification for access instructions.

LENGTHY TABLE REFERENCED HERE
US20070015148A1-20070118-T00004
Please refer to the end of the specification for access instructions.

LENGTHY TABLE REFERENCED HERE
US20070015148A1-20070118-T00005
Please refer to the end of the specification for access instructions.

LENGTHY TABLE
The patent application contains a lengthy table section. A copy of the table is available in electronic form from the USPTO web site (http://seqdata.uspto.gov/?pageRequest=docDetail&DocID=US20070015148A1) An electronic copy of the table will also be available from the USPTO upon request and payment of the fee set forth in 37 CFR 1.19(b)(3).

What is claimed is:

1. A method of diagnosing breast cancer in a patient, comprising:
(a) detecting the level of expression in a tissue sample of two or more genes from Tables 1-5; wherein differential expression of the genes in Tables 1-5 is indicative of breast cancer.

2. A method of detecting the progression of breast cancer in a patient, comprising:
(a) detecting the level of expression in a tissue sample of two or more genes from Tables 1-5; wherein differential expression of the genes in Tables 1-5 is indicative of breast cancer progression.

3. A method of monitoring the treatment of a patient with breast cancer, comprising:
(a) administering a pharmaceutical composition to the patient;
(b) preparing a gene expression profile from a cell or tissue sample from the patient; and
(c) comparing the patient gene expression profile to a gene expression from a cell population selected from the group consisting of normal breast cells and cancerous breast cells.

4. A method of treating a patient with breast cancer, comprising:
(a) administering to the patient a pharmaceutical composition, wherein the composition alters the expression of at least one gene in Tables 1-5;
(b) preparing a gene expression profile from a cell or tissue sample from the patient comprising tumor cells; and
(c) comparing the patient expression profile to a gene expression profile selected from the group consisting of normal breast cells and cancerous breast cells.

5. A method of typing breast cancer in a patient, comprising:
(a) detecting the level of expression in a tissue sample of two or more genes from Tables 1-5; wherein differential expression of the genes in Tables 1-5 is indicative of a type of breast cancer selected from a group consisting of infiltrating ductal carcinoma, microinvasive carcinoma, cribiform carcinoma, stage I carcinoma, stage II carcinoma, stage III carcinoma or lobular carcinoma.

6. A method of detecting the presence or progression of infiltrating ductal carcinoma in a patient, comprising:
(a) detecting the level of expression in a tissue sample of two or more genes from Tables 1-5; wherein differential expression of the genes in Tables 1-5 is indicative of infiltrating ductal carcinoma progression.

7. A method of monitoring the treatment of a patient with infiltrating ductal carcinoma, comprising:
(a) administering a pharmaceutical composition to the patient;
(b) preparing a gene expression profile from a cell or tissue sample from the patient; and
(c) comparing the patient gene expression profile to a gene expression from a cell population comprising normal breast cells or to a gene expression profile from a cell population comprising infiltrating ductal carcinoma cells or to both.

8. A method of treating a patient with infiltrating ductal carcinoma, comprising:
(a) administering to the patient a pharmaceutical composition, wherein the composition alters the expression of at least one gene in Tables 1-5;
(b) preparing a gene expression profile from a cell or tissue sample from the patient comprising infiltrating ductal carcinoma cells; and
(c) comparing the patient expression profile to a gene expression profile from an untreated cell population comprising infiltrating ductal carcinoma cells.

9. A method of diagnosing a microinvasive form of breast tumor in a patient, comprising:
(a) detecting the level of expression in a tissue sample of two or more genes from Tables 1-5; wherein differential expression of the genes in Tables 1-5 is indicative of a microinvasive form of breast cancer.

10. A method of detecting the progression of a microinvasive for of breast cancer in a patient, comprising:
(a) detecting the level of expression in a tissue sample of two or more genes from Tables 1-5; wherein differential expression of the genes in Tables 1-5 is indicative of the progression of a microinvasive form of breast cancer.

11. A method of monitoring the treatment of a patient with a microinvasive form of breast cancer, comprising:
(a) administering a pharmaceutical composition to the patient;
(b) preparing a gene expression profile from a cell or tissue sample from the patient; and
(c) comparing the patient gene expression profile to a gene expression from a cell population comprising normal breast cells or to a gene expression profile from a cell population comprising microinvasive breast cancer cells or to both.

12. A method of treating a patient with a microinvasive form of breast cancer, comprising:
(a) administering to the patient a pharmaceutical composition, wherein the composition alters the expression of at least one gene in Tables 1-5;
(b) preparing a gene expression profile from a cell or tissue sample from the patient comprising microinvasive breast cancer cells; and
(c) comparing the patient expression profile to a gene expression profile from an untreated cell population comprising microinvasive breast cancer cells.

13. A method of differentiating microinvasive breast cancer from a benign growth in a patient, comprising:
(a) detecting the level of expression in a tissue sample of two or more genes from Tables 1-5; wherein differential expression of the genes in Tables 1-5 is indicative of microinvasive breast cancer rather than benign growth.

14. A method of screening for an agent capable of modulating the onset or progression of breast cancer, comprising:
(a) preparing a first gene expression profile of a cell population comprising breast cancer cells, wherein the expression profile determines the expression level of one or more genes from Tables 1-5;
(b) exposing the cell population to the agent;
(c) preparing second gene expression profile of the agent-exposed cell population; and
(d) comparing the first and second gene expression profiles.

15. The method of claim 14, wherein the breast cancer is a infiltrating ductal carcinoma.

16. The method of claim 14, wherein the breast cancer is a microinvasive breast cancer.

17. A composition comprising at least two oligonucleotides, wherein each of the oligonucleotides comprises a sequence that specifically hybridizes to a gene in Tables 1-5.

18. A composition according to claim 17, wherein the composition comprises at least 3 oligonucleotides.

19. A composition according to claim 17, wherein the composition comprises at least 5 oligonucleotides.

20. A composition according to claim 17, wherein the composition comprises at least 7 oligonucleotides.

21. A composition according to claim 17, wherein the composition comprises at least 10 oligonucleotides.

22. A composition according to any one of claims 17-21, wherein the oligonucleotides are attached to a solid support.

23. A composition according to claim 22, wherein the solid support is selected from a group consisting of a membrane, a glass support, a filter, a tissue culture dish, a polymeric material, a bead and a silica support.

24. A solid support comprising at least two oligonucleotides, wherein each of the oligonucleotides comprises a sequence that specifically hybridizes to a gene in Tables 1-5.

25. A solid support according to claim 24, wherein the oligonucleotides are covalently attached to the solid support.

26. A solid support according to claim 24, wherein the oligonucleotides are non-covalently attached to the solid support.

27. A solid support according to claim 24, wherein the support comprises at least about 10 different oligonucleotides in discrete locations per square centimeter.

28. A solid support according to claim 24, wherein the support comprises at least about 100 different oligonucleotides in discrete locations per square centimeter.

29. A solid support according to claim 24, wherein the support comprises at least about 1000 different oligonucleotides in discrete locations per square centimeter.

30. A solid support according to claim 24, wherein the support comprises at least about 10,000 different oligonucleotides in discrete locations per square centimeter.

31. A computer system comprising:
(a) a database containing information identifying the expression level in breast tissue of a set of genes comprising at least two genes in Tables 1-5; and
(b) a user interface to view the information.

32. A computer system of claim 31, wherein the database further comprises sequence information for the genes.

33. A computer system of claim 31, wherein the database further comprises information identifying the expression level for the genes in normal breast tissue.

34. A computer system of claim 31, wherein the database further comprises information identifying the expression level for the genes in breast cancer tissue.

35. A computer system of claim 34, wherein the breast cancer tissue comprises infiltrating ductal carcinoma cells.

36. A computer system of claim 34, wherein the breast cancer tissue comprises microinvasive breast cancer cells.

37. A computer system of any of claims 31-36, further comprising records including descriptive information from an external database, which information correlates said genes to records in the external database.

38. A computer system of claim 37, wherein the external database is GenBank.

39. A method of using a computer system of any one of claims 31-36 to present information identifying the expression level in a tissue or cell of at least one gene in Tables 1-5, comprising:
(a) comparing the expression level of at least one gene in Tables 1-5 in the tissue or cell to the level of expression of the gene in the database.

40. A method of claim 39, wherein the expression level of at least two genes are compared.

41. A method of claim 39, wherein the expression level of at least five genes are compared.

42. A method of claim 39, wherein the expression level of at least ten genes are compared.

43. A method of claim 39, further comprising displaying the level of expression of at least one gene in the tissue or cell sample compared to the expression level in breast cancer.

44. A kit comprising at least one solid support of any one of claims 24-30 packaged with gene expression information for said genes.

45. A kit of claim 44, wherein the gene expression information comprises gene expression levels in a breast cancer tissue or cell sample.

46. A kit of claim 45, wherein the gene expression information is in an electronic format.
